(19) United States
(12) Patent Application Publication
(10) Pub. No.: US 2005/0053648 A1
(43) Pub. Date: Mar. 10, 2005

(54) Medication delivery device

(76) Inventor: Anne Marie Chalmers, Osprey, FL (US)

Correspondence Address: LAHIVE & COCKFIELD, LLP., 28 STATE STREET, BOSTON, MA, 02109, US

(21) Appl. No.: 10/657,521

(22) Filed: Sep. 8, 2003

(51) Int. Cl. A61K 9/48

(52) U.S. Cl. 424/451

(57) ABSTRACT

A medication delivery device by which multiple medications are encased within separate containers so that each of the said medications are not in actual physical contact with each other until the said containers degrade within a live body, with said separate containers being themselves embedded in a series of such containers, that is, containers within containers, which containers are biodegradable within a live body.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE OR A COMPUTER PROGRAM LISTING COMPACT DISK APPENDIX

Not Applicable

### BACKGROUND OF INVENTION

This invention relates to a device for the simultaneous delivery into a live body of multiple medication products, pharmaceuticals, nutritional products and inert materials.

As used herein, the phrase “pill”, “capsule”, and “soft-gel” are used interchangeably, unless specifically otherwise limited in scope at a specific section herein, to include and encompass, and not necessarily be limited to “pills and capsules and soft-gels”, and indeed encompass all other mechanisms and means for delivery of medication products into a live body.

As used herein, the phrase “medication product” includes and encompasses, but is not limited to, prescription drugs, non-prescription drugs, over-the-counter drugs, nutritional supplements and inert “filler” materials.

As used herein, the phrases “container” and “containment means” includes and encompasses not only the traditional medication capsule, pill, gel cap, suppositories, skin patches and sublingual applications, but also includes and encompasses any and all other medication delivery mechanisms.

As used herein, the phrase “outer shell” means the container or containment means which itself is not contained within further container or containment means, which further container or containment means is intended for ingestion or insertion into a live body.

Due to an increasing aging population and increased use of drug therapy, more and more people find themselves taking several pills, tablets or capsules for treating or preventing illnesses every day. Research has shown that even patients for whom strict adherence to prescribed drug regimens is crucial, rates of non-compliance can still range from as much as 20% to 50%.

Each year in the United States, the consequences of poor compliance cost an estimated $100 billion in added health care expenses, lost productivity, and other direct and indirect costs, in addition to personal suffering.

One means of increasing compliance is to reduce the number of pills taken per day, thus reducing patient resistance to swallowing large numbers of pills or the possibility of patients forgetting to take some of their medication.

Moreover, numerous studies have shown that certain combinations of different substances or medications can dramatically improve the health outcomes through additional or synergic effects. But these combinations most often require the ingestion of more pills which will again lower the compliance.

The delivery of medication products, including prescription drugs, over-the-counter drugs, nutritional supplements and inert materials, has been traditionally accomplished by the use of pills, capsules, and gel caps.

Typically pills are comprised of the active ingredient compounded with inert ingredients for various purposes, including ease of handling small amounts of active ingredients, with that mixture of active and inactive ingredients being then compressed to form a “pill”. Very often medications can come in a dose of only a few milligrams, but since this amount is so small and unmanageable, the size of the pill has to be increased substantially with fillers

Similarly, capsules and gel caps are composed of an outer material or casing which is dissolved after ingestion by the patient, with the interior portion of the capsule or gel cap being filled with an active ingredient compounded with inert ingredients for various purposes, including ease of handling of small amounts of active ingredients, similar to a pill.

There are several distinctions between capsules vis-a-vis soft gels or gel caps. The major difference between a capsule and a soft gel or gel cap is that a capsule is a hard shell and a soft gel or gel cap is physically a softer gelatin container. A soft gel or gel cap is essentially a capsule made from gelatin, usually from a bovine or pig source, although there are also available soft gels made from vegetable sources, e.g., potato starch, whereas a capsule can be generally be made from many different materials, including gelatin formulations. Generally capsules usually contain solid materials such as powders, although they occasionally do contain liquids, whereas soft gels or gel caps usually contain oils or liquid, although some soft gels or gel caps do contain rather powdery substances in soft gels or gel caps, which facilitate the swallowing process.

A sub-category or variation of gel caps is “liquid gel caps”, which are gel caps, the interior of which are filled with liquid rather than solid materials, typically a blend of active and inactive ingredients. The liquid gel caps provide an easy means of carrying liquid medications for ingestion without the need to transport the liquid from a liquid container to the mouth of the patient, thus simultaneously avoiding the risk of liquid bottle spillage or breakage.

The disadvantage of a typical prescription pill, capsule or gel cap is that they usually contain only one primary medication thus only addressing one type of indication or problem.

The treatment of many afflictions and diseases requires the use of multiple medications by the respective patient, as to which the use of numerous traditional pills, capsules or gel caps is burdensome. Not only does the patient need to be burdened with multiple containers for the various medications, but the patient must also track to assure that they have in fact timely ingested the proper dosage of each such medication.

An objective of the present invention is to solve the aforesaid problems, including by reducing the number of pills that will contain the originally intended, prescribed or recommended medications and doses, thus increasing compliance.

### BRIEF SUMMARY OF THE INVENTION

A medication delivery device by virtue of which multiple medications are encased within separate containers so that each of the said medications are not in actual physical contact with each other (except arguably in the instance of uncoated pills) until the said containers degrade within a live body, with said separate containers being themselves embedded in a series of such containers, that is, containers within containers, which containers are biodegradable within a live body.

One of several preferred embodiments of the invention is a capsule within a capsule, wherein the first and smaller capsule is filled with a first medication, with that first capsule then itself contained within a second capsule, with that second capsule containing not only the first capsule but also a second medication in which the first capsule is immersed.

With the use of the present invention, by virtue of the active substances in the medications being contained within an outer shell or container, problems normally encountered by patients' dislike of the aroma or taste of certain medications are eliminated.

Furthermore, by virtue of the invention providing for separate sequestered compartments or containers or containment means within a larger container, containment means or outer shell, the invention provides for combining liquid medications with solid medications.

### BRIEF DESCRIPTION OF THE DRAWINGS

FIG. 1 is a cross-section of a preferred embodiment of the invention.

FIG. 2 is a cross-section of a second preferred embodiment of the invention.

FIG. 3 is a cross-section of a third preferred embodiment of the invention

FIG. 4 is a cross-section of a fourth preferred embodiment of the invention.

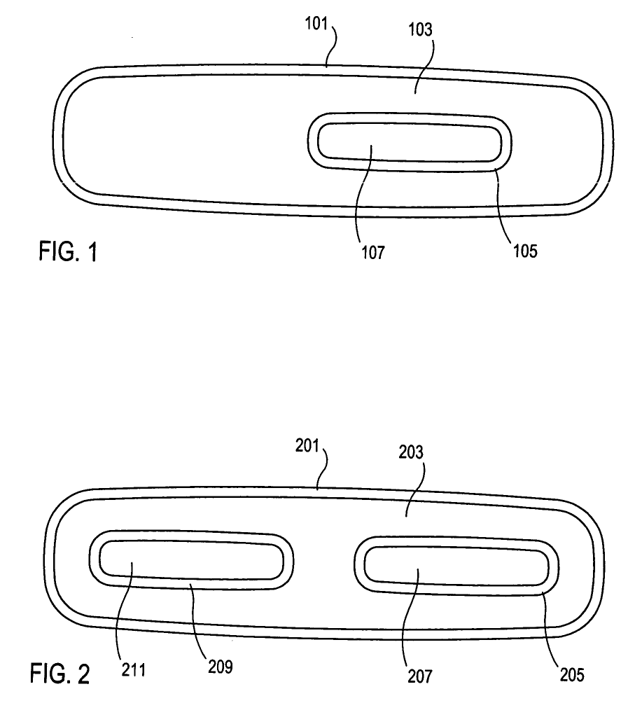

These FIGS. 1 through FIG. 4 are not necessarily exhaustive of all embodiments of the invention.

### DETAILED DESCRIPTION OF THE INVENTION

A medication delivery device in accordance, as exemplified by the several preferred embodiments depicted in FIGS. 1, 2, 3 and 4, provides for the delivery to a living body, human or animal, of multiple medication products simultaneously.

Although the more typical method and means for entry into the said living body is by ingestion, the invention also encompasses other forms of such entry, including but not limited to suppository form or application to the skin.

As depicted in FIG. 1, one preferred embodiment of the invention is comprised of a capsule (105) within a capsule (101), wherein the first and smaller capsule (105) is filled with a first medication (107), with that first capsule (105) then itself contained within a second capsule (101), with that second capsule (101) containing not only the first capsule (105) but also a second medication (107) in which the first capsule is immersed.

The first medication is pharmacologically distinct from the second medication.

The first medication may function in a synergistically physiological manner in conjunction with the second medication.

For example, the first medication may be a prescription pharmaceutical and the second medication may be a non-prescription nutritional supplement which has been clinical established as enhancing the efficacy of the first medication.

Alternatively, there need not be any interaction between the first medication and the second medication. For example each medication can have separate and different effects on the body.

As depicted in FIG. 2, a second preferred embodiment of the invention is comprised of a first capsule (201) which contains within it a second capsule (205), and also contains within said first capsule (201) a third capsule (209), wherein the void within the said first capsule (201) not occupied by the second capsule (205) and the third capsule (209) is filled with a first medication (203), and said second capsule is filled with a second medication (207) and said third capsule is filled with a third medication (211).

The first medication (203), the second medication (207) and the third medication (211) are each pharmacologically distinct from each other.

The first medication (203), the second medication (207) and the third medication (211) may function in a synergistically physiological manner in conjunction with the either of the other two medications, or with both of them.

For example, the first medication may be a prescription pharmaceutical and the second medication may be a non-prescription nutritional supplement which has been clinical established as enhancing the efficacy of the first medication.

Alternatively, the first medication (203), the second medication (207) and the third medication (211) may not function in a synergistically physiological manner in conjunction with either of the other two medications, or with both of them, each having their own indications.

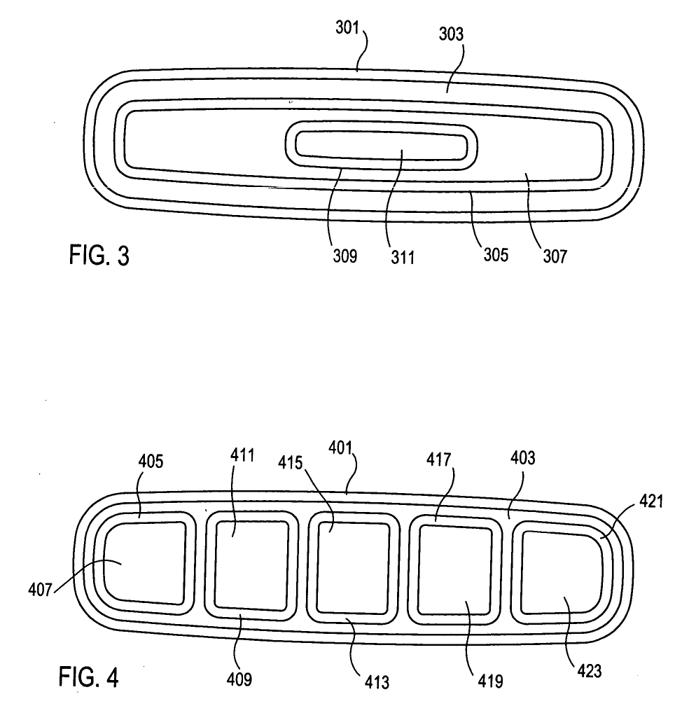

Further, as depicted in FIG. 3, a third preferred embodiment of the invention is comprised of a first capsule (301) which contains within it a second capsule (305), with the said second capsule (305) containing within it (305) a third capsule (309), wherein the void within the said first capsule (301) which is not occupied by the second capsule (305) is filled with a first medication (303), and wherein the void within the said second capsule (305) which is not occupied by the third capsule (309) is filled with a second medication (307), and said third capsule (309) is filled with a third medication (311).

The first medication (303), the second medication (307) and the third medication (311) are each pharmacologically distinct from each other.

The first medication (303), the second medication (307) and the third medication (311) may function in a synergistically physiological manner in conjunction with either of the other two medications, or with both of them.

For example, the first medication may be a prescription pharmaceutical and the second medication may be a non-prescription nutritional supplement which has been clinically established as enhancing the efficacy of the first medication(for example statins and omega-3).

Alternatively, the first medication (303), the second medication (307) and the third medication (311) may not function in a synergistically physiological manner in conjunction with the either of the other two medications, or with both of them, or compliment each other (for example aspirin and blood pressure medication) or may have completely different indications (for example anti-depressants and statins).

FIG. 4 is a depiction of a fourth preferred embodiment of the invention, which is comprised of a first capsule (401) which contains within it multiple additional capsules (405), (409), (413), (417) and (421), wherein the void within the said first capsule (401) which is not occupied by the said multiple additional capsules (405), (409), (413), (417) and (421), is filled with a first medication (403), with the said multiple additional capsules (405), (409), (413), (417) and (421), being filled with medications (407), (411), (415), (419) and (423), respectively.

Each of the multiple additional capsules (405), (409), (413), (417) and (421), are shaped and configured so that in relation to the additional internal container(s) to which they are contiguous when enclosed within the outermost container (401), the said outermost container (401) is thus enabled to contain an optimally high number and density of multiple medications in a minimally sized outermost container (401).

Each of the medications (403), (405), (409), (413), (417) and (421) are pharmacologically distinct from each other.

Each of the medications (403), (405), (409), (413), (417) and (421) may function in a synergistically physiological manner in conjunction with the any, some or all of the other medications contained (separately or otherwise) within the outermost container (401).

Although the foregoing embodiments refer to capsules, the invention is not limited to capsules, but rather encompasses any and all medication containers and containment means, including capsules, but also including but not limited to gel caps and pills as discussed herein.

In addition, although several of the preferred embodiments described hereinbefore are comprised, for illustration purposes, of only one or two capsules internal to the outer shell (or in the instance of FIG. 4, five internal capsules), the invention is not limited to a specific number of such internal capsules, but rather encompasses any number of internal containers or containment means.

A very small list of numerous examples of types and categories of medication products which would benefit from the use of this invention include: (a) statin+aspirin; (b) Statin+aspirin+omega-3; and (c) Statin+aspirin+omega 3+a blood pressure medication; and (d) fat soluble essential element+water soluble element (such as omega 3+vitamin B complex).

Each container compartment is separated from the other ones by a distinct barrier or wall, preventing the medication substance in one container compartment from mixing with the medication substance in the other container compartments.

Consequently, there is no concern about the medication substances starting to react with each other within the most outer container compartment, and the medications substances will therefore not represent a new chemical compound, before being ingested or otherwise delivered into a live body. Furthermore, by virtue of the container compartments being “separate chambers”, the highly economically attractive and flexible prospect of different medication substances actually being produced at locations distant from each other and then being consolidated in medication delivery devices in accordance with this invention become feasible.

The numerous advantages of this invention include: (a) The ability to maintain the chemical stability of the different medication substances, by preventing any chemical reaction between or among them by virtue of the fact that they are separated from each other by the inert wall which is the container compartment; (b) Ease of production of a means to delivery multiple medications simultaneously, by virtue of each container compartment being capable of being produced and filled with different medications at remote distant locations, before the final assemblage; (c ) Flexibility, since the content or concentration can be changed for one substance without influencing the chemical properties of the other; (d) Increased intestinal absorption, since the bio-availability is usually higher for soft gels and capsules compared to tablets or pills; (e) Increased patient compliance and assurance that the patient is actually taking several medical substances since they come as “one dose”; (f) Increased patient compliance because the patient will be more willing to take one pill compared to several; (g) A high patient compliance because patients often have an easier time swallowing a capsule or soft gel or gel cap compared to a tablet or pill; (h) A precise “medical” communication, because it shows clearly which products are combined (not mixed), and thus opening the area of “synergy” medicine.

It is contemplated that the inventive concepts herein described may be variously otherwise embodied and it is intended that the appended claims be construed to include alternative embodiments of the invention except only insofar as limited by prior art.

1. A device for the simultaneous delivery of multiple medication products to a live body, with such device comprising:
a first dosage of a first medication product contained within a first containment means;
a second dosage of a second medication product contained within a second containment means; and
the first containment means itself contained within the second containment means.

2. A device for the simultaneous delivery of multiple medication products to a live body, with such device comprising:
a first dosage of a first medication product contained within a first containment means;
a second dosage of a second medication product contained within a second containment means;
a third dosage of a third medication product contained within a third containment means; and
the first containment means itself containing the second containment means and the third containment means immersed in the said first dosage of said first medication product which is also contained within the first containment means.

3. A device for the simultaneous delivery of multiple medication products to a live body, with such device comprising:
a first dosage of a first medication product contained within a first containment means;
a second dosage of a second medication product contained within a second containment means;
a third dosage of a third medication product contained within a third containment means;
the first containment means itself containing the second containment means;
the second containment means itself containing the third containment means;
the second containment means immersed in the said first dosage of said first medication product which is also contained within the first containment means; and
the third containment means immersed in the said second dosage of said second medication product which is also contained within the second containment means.

4. A device for the simultaneous delivery of multiple medication products to a live body, with such device comprising:
a first dosage of a first medication product contained within a first containment means;
a second dosage of a second medication product contained within a second containment means;
a third dosage of a third medication product contained within a third containment means;
the first containment means itself containing the second containment means and the third containment means immersed in the said first dosage of said first medication product which is also contained within the first containment means; and
the second containment means and the third containment means being shaped and configured so that in relation to each other when they are contiguously positioned within the first containment means, the said first containment means is thus enabled to contain an optimally high number and density of multiple medications in a minimally sized first container means.

5. A device for the simultaneous delivery of multiple medication products to a live body, with such device comprising:
a first dosage of a first medication product contained within a first pill, capsule or gel cap;
a second dosage of a second medication product contained within a second pill, capsule or gel cap; and
the said first pill, capsule or gel cap itself contained within the said second pill, capsule or gel cap.

6. A device for the simultaneous delivery of multiple medication products to a live body, with such device comprising:
a first dosage of a first medication product contained within a first pill, capsule or gel cap;
a second dosage of a second medication product contained within a second pill, capsule or gel cap;
a third dosage of a third medication product contained within a third pill, capsule or gel cap; and
the said first pill, capsule or gel cap itself containing the second pill, capsule or gel cap and the third pill, capsule or gel cap immersed in the said first dosage of said first medication product which is also contained within the first pill, capsule or gel cap.

7. A device for the simultaneous delivery of multiple medication products to a live body, with such device comprising:
a first dosage of a first medication product contained within a first pill, capsule or gel cap;
a second dosage of a second medication product contained within a second pill, capsule or gel cap;
a third dosage of a third medication product contained within a third pill, capsule or gel cap;
the first pill, capsule or gel cap itself containing the second pill, capsule or gel cap;
the second pill, capsule or gel cap itself containing the third pill, capsule or gel cap;
the second pill, capsule or gel cap immersed in the said first dosage of said first medication product which is also contained within the first pill, capsule or gel cap; and
the third pill, capsule or gel cap immersed in the said second dosage of said second medication product which is also contained within the second pill, capsule or gel cap.

8. A device for the simultaneous delivery of multiple medication products to a live body, with such device comprising:
a first dosage of a first medication product contained within a first pill, capsule or gel cap;
a second dosage of a second medication product contained within a second pill, capsule or gel cap;
a third dosage of a third medication product contained within a third pill, capsule or gel cap;
the first pill, capsule or gel cap itself containing both the second pill, capsule or gel cap and the third pill, capsule or gel cap immersed in the said first dosage of said first medication product which is also contained within the first pill, capsule or gel cap; and
the second pill, capsule or gel cap and the third pill, capsule or gel cap being shaped and configured so that in relation to each other when they are contiguously positioned within the first pill, capsule or gel cap, the said first pill, capsule or gel cap is thus enabled to contain an optimally high number and density of multiple medications in a minimally sized first pill, capsule or gel cap.
